(19) United States
(12) Patent Application Publication
(10) Pub. No.: US 2021/0379256 A1
(43) Pub. Date: Dec. 9, 2021

(54) Total Heart Assistance Device

(71) Applicant: Samuel Gueller, Cincinnati, OH (US)

(72) Inventor: Samuel Gueller, Cincinnati, OH (US)

(21) Appl. No.: 16/893,161

(22) Filed: Jun. 4, 2020

(51) Int. Cl. A61M 1/10

(52) U.S. Cl. CPC A61M 1/1086; A61M 1/1096; A61M 2210/125; A61M 2209/08; A61M 1/1065

(57) ABSTRACT

The present invention relates generally to the field of cardiac, vascular system, and heart assistance devices. It provides the energy required to keep the blood flowing in the pulmonary and systemic circuits to a desired level, acting on one or more chambers. Actual problems of Total Artificial Heart pumping blood are design limitations, infection, hemorrhage, end organ failure, thromboembolism, device disfunction, life span of diaphragms, and impossibility to restore the heart but with a transplant. The device is external and has four units replicating the natural heart and its dynamics, driving by a pneumatic transcutaneous system to provide the energy needed up to the desired working level of a healthy organ. Applications are on those types of surgical or clinical treatment of patients with Diastolic Heart Failure or used to treat Heart Failure with Reduced Ejection Fraction (Systolic Heart Failure), the device can be left connected permanently of for healing.

### FIELD OF THE INVENTION

The present invention relates generally to the field of cardiac, vascular system, and heart assistance devices. Applications are in those types of surgical or clinical treatment where a total or partial prosthesis is required.

### BACKGROUND OF THE INVENTION

One of the main medical problems is characterized by the inability of the left ventricle to relax properly and fill with blood. This is caused by the stiffening and impaired relaxation with normal systolic function, either due to hypertrophy or to processes such as fibrosis and infiltrative diseases. These changes cause high LV filling pressures, leading to pulmonary congestion and atrial fibrillation due to distention of the atrium. The treatment of patients with this named Diastolic Heart Failure is mainly empirical. The treatment includes modification of the underlying risk factors for the disease (such as hypertension and diabetes) and administration of medications used to treat Heart Failure with reduced Ejection Fraction (Systolic Heart Failure). To address this problem normal pressure in the four chambers of the heart is required and in the 1960s the first intent to build a total artificial heart (TAH) was by means of chambers where elastic rubber forced blood to flow under the impulse provided by an external fix compressor's air. In the early 1980s an electric device was borne in concept, in practice an operating one was possible in the middle 2000 at a great effort and cost, but with same performance as the previous ones, or about one-year survival time on the average, which hasn't increase since then. While considerable strides have been made in design of a TAH, two main obstacles to clinical success stem from the continued thrombogenic nature of the materials employed in the devices, coupled with design limitations. The surfaced thrombogenicity necessitates systemic antithrombotic therapy with the attendant risk of hemorrhage. Mayor remaining clinical complications with the TAH include infection, hemorrhage, end organ failure, thromboembolism and device dysfunction. Mayor causes of death with current TAH designs include sepsis, multi-organ failure, neurological death most likely due to thromboembolism, hemorrhage and problems with device fit. Thus, there is an actual need to overcome those problems by leaving the heart intact and use removable means as a partial or total artificial heart where the four chambers get a complementary help to function satisfactorily by an external console to manage the necessary beats per minute and blood pressure in the circulatory system.

### SUMMARY OF THE INVENTION

External hardware out of a patient body to provide pneumatic energy to accompany the heart wall's movements under cardiac insufficiency, up to the condition of normal functioning following the dynamics of those walls with proper set up and automation.

### BRIEF DESCRIPTION OF THE DRAWINGS

FIG. 1A is a general view with the patient's component and external hardware, where the Console is synonyms of panel and contain a Control Box. FIG. 1B shows the components installed in a belt for patient's freedom of movements and quality of life.

FIG. 2.A is a diagram to visualize air flow in the begin of Systole to assess which valves are closed or open in the sequencing dynamics when a Basic Unit works. FIG. 2.B is a diagram to visualize air flow in the end of Systole to assess which valves are closed or open in the sequencing dynamics when a Basic Unit works. FIG. 2.C is a diagram to visualize air flow in the begin of Diastole to assess which valves are closed or open in the sequencing dynamics when a Basic Unit works. FIG. 2.D is a diagram to visualize air flow in the end of Diastole to assess which valves are closed or open in the sequencing dynamics when a Basic Unit works.

FIG. 3.A is the dependent unique symbol of a continuously differentiable servo proportional valve. FIG. 3.B shows the schematic electric lines and minimum switches and alarms components installed in a belt. FIG. 3.C show a Basic Unit (e.g.: left ventricle) locating the 6 valves, power in, and air in and out. There are four interconnected basic units thru the Control Box in the Console.

FIG. 4 Diagram to analyze air pressure added and released to reach the optimum level of functioning.

### DETAILED DESCRIPTION OF THE DRAWINGS

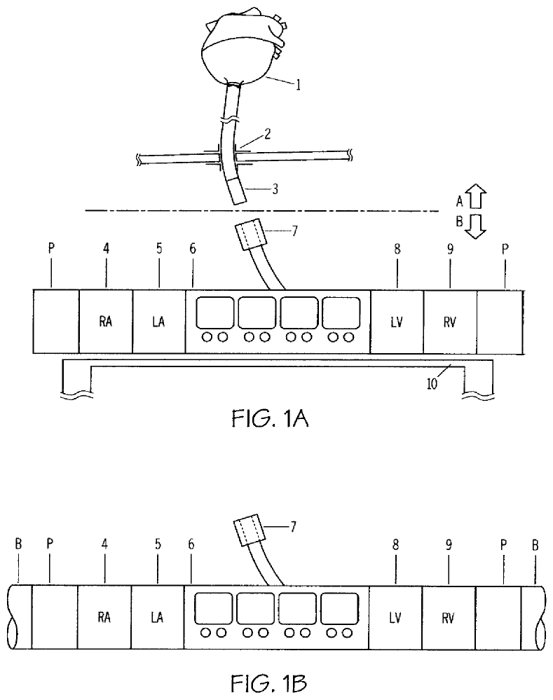

FIG. 1A in this diagram of a typical attachment number identifies the following parts:

FIG. 1.A 1: Bi-ventricle advanced active cuff

FIG. 1.A 2: Transcutaneous Attachment

FIG. 1.A 3: Fastener connector male Coupling Nuts

A: Denote to belong in the Public Domain

B: Denote to the Claims of this application

FIG. 1.A 4: RA, Right Atria Unit

FIG. 1.A 5: LA, Left Atria Unit

FIG. 1.A 6: Main Console

FIG. 1.A 7: Fastener connector female Coupling Nuts

FIG. 1.A 8: LV, Left Ventricle Unit

FIG. 1.A 9: RV, Right Ventricle Unit

FIG. 1.A P: Power Source

FIG. 1.A 10: Tabletop or Cart

FIG. 1B in this diagram of a typical attachment numbers identifies the following parts:

FIG. 1.B B: Belt

FIG. 1.B P: Power Source

FIG. 1.B 4: Right Atria Unit

FIG. 1.B 5: Left Atria Unit

FIG. 1.B 6: Main Console

FIG. 1.B 7: Fastener connector female

FIG. 1.B 8: Left Ventricle Unit

FIG. 1.B 9: Right Ventricle Unit

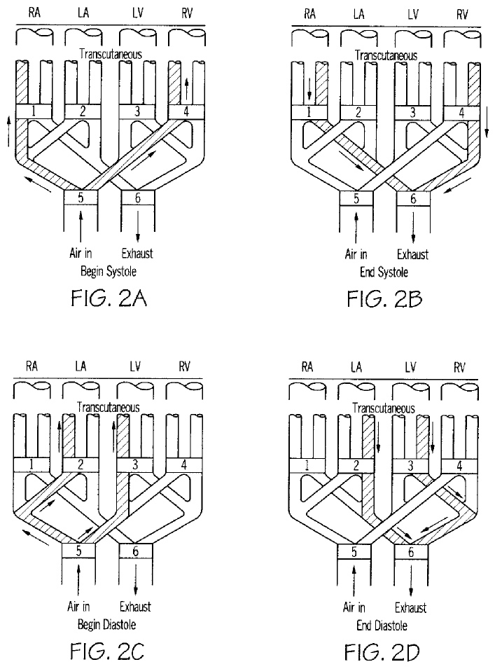

FIG. 2A in this diagram of a typical attachment numbers identifies the following parts:

FIG. 2.A 1—RA, Right Atria Proportional Valve

FIG. 2.A 2—LA, Left Atria Proportional Valve

FIG. 2.A 3—LV, Left Ventricle Proportional Valve

FIG. 2.A 4—RV, Right Ventricle Proportional Valve

FIG. 2.A 5—Air in Proportional Valve

FIG. 2.A 6—Exhaust Proportional Valve

FIG. 2B in this diagram of a typical attachment numbers identifies the following parts:

FIG. 2.B 1—RA, Right Atria Proportional Valve

FIG. 2.B 2—LA, Left Atria Proportional Valve

FIG. 2.B 3—LV, Left Ventricle Proportional Valve

FIG. 2.B 4—RV, Right Ventricle Proportional Valve

FIG. 2.B 5—Air in Proportional Valve

FIG. 2.B 6—Exhaust Proportional Valve

FIG. 2C in this diagram of a typical attachment numbers identifies the following parts:

FIG. 2.C 1—RA, Right Atria Proportional Valve

FIG. 2.C 2—LA, Left Atria Proportional Valve

FIG. 2.C 3—LV, Left Ventricle Proportional Valve

FIG. 2.C 4—RV, Right Ventricle Proportional Valve

FIG. 2.C 5—Air in Proportional Valve

FIG. 2.C 6—Exhaust Proportional Valve

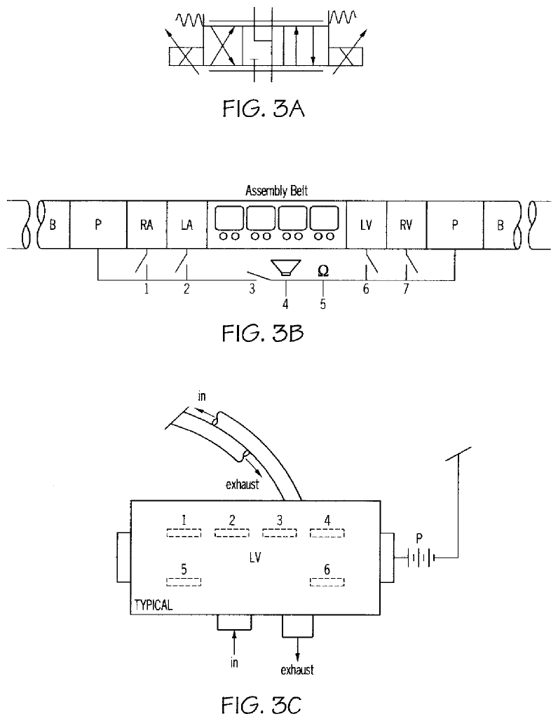

FIG. 3D in this diagram of a typical attachment numbers identifies the following parts:

FIG. 2.D 1—RA, Right Atria Proportional Valve

FIG. 2.D 2—LA, Left Atria Proportional Valve

FIG. 2.D 3—LV, Left Ventricle Proportional Valve

FIG. 2.D 4—RV, Right Ventricle Proportional Valve

FIG. 2.D 5—Air in Proportional Valve

FIG. 2.D 6—Exhaust Proportional Valve

### DETAILED DESCRIPTION OF THE INVENTION

These heart attachments are activated by compressed air from a pneumatic source, where the potential energy applied produce expansive work against the heart walls. The air flow is different for each patch to mimic or follow the total or remaining dynamic of the natural heart with which they interact fully. The compressed air fills a pocket, pouch, as a patch to add partial, or total, force such that the atria or ventricle complete its work efficiently providing blood flow, pressure and pulsation across the vascular system to reach those points in the body that require them. In doing so, strict maximum and minimum limits are followed not to damage he heart itself or any part of the systemic or pulmonary system or irrigated tissues or organs.

FIG. 1A and FIG. 1B shows how the installation will look like before it operates. Previous work includes surgery for pass-through the skin the connecting pipes to the external pneumatic unit, control box and panel, as well as open chest to access the beating heart directly. Preparation of the patient and the attachment itself are: Use of ultrasound imagens for 3D dimensioning the heart at its maximum volume to produce a solid model to serve as support to put together the membranes and layers. The whole attachment added power to a failing heart requires a carefully synchronization with the beating heart without stopping it, this is done by watching the ultrasound monitor dynamics, reading real time electrograph, pulse, max/min pressure before beginning to add slowly extra force and contraction/expansion up to an optimum point adjusting for optimization, using valves and servomechanisms only, always from low to high values.

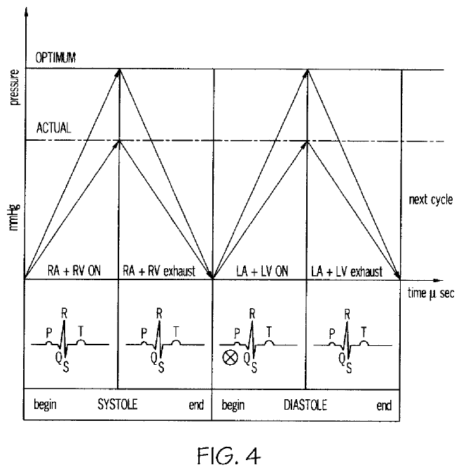

Regime is obtained by the servo valves. The console's units mimic the natural heart 2 atria versus 2 ventricles, or any other configuration. FIG. 4 shows sequencing of filling and emptying the chambers, thou they may be partial, complementary, or total in an inactive heart when the case would be a total artificial heart (TAH) assistance device without removal and replace of the human heart.

The Control Panel can be replaced easily by a new one at any time in seconds if there is remaining activity in the patient heart. The Control Panel can operate under water as if in an accident or sport, in this case the Panel must be waterproofed, and with a pouch of air instead of the air circuit connected to the atmosphere in the pneumatic unit.

In and out of the assistance to a beaten natural heart: The in-operation is done by looking at the pressure gages of the patient and the output from the pneumatic unit in the control external box, the reading at the patient most probably is lower than normal, then a single valve is activated in the control box to start from zero to add pressure to the wall of the failing chamber, atria or ventricle, slowly. Continuing sequentially with the other three (if needed) chambers. The opposite will be done if the intention is an out-operation, receding to zero the reading in the control box. Once completed the failing heart will work as a failing organ, until reconnecting the control box. DANGER: If pressure is applied and the patient pressure didn't rise, is negative or is zero, means that such applied pressure work against the heart and must be stopped immediately thou slowly. When all chambers are set to work conveniently, they are put fix to provide the delivered assistance. Only a trained professional is responsible to made changes thereof, by operating the governing valves, for a new assessment and set up the operation properly or disconnect the control box, if applicable.

Security and alarms for emergencies has been taken in consideration, in particular stop by run out of range of pre-stablished and loss of air into the thorax cavity.

This device though for final use in humans, begin as a tool for medical researches to test patches and setting them up in place, which may deserve an additional patent.

This console works independent of a complete Total Heart Assistance Device but could be used as an external one, any suggestion of a patch system cannot be used in the US or other Countries that are not permitted under local regulations.

If the law permit can be used for studies in open chest animals under anesthesia and closing chest without sacrificing the animal.

Fundamentals: To work this device in accord with the heart, to complement its function if necessary, we need to analyze how the heart works from a physics point of view. Blood circulate in vessels and tissues constantly under steady state and the heart provide the dynamics for that to happen. The heart by itself expend kinetic energy for that purpose and return to a potential level above the level of a ground zero, which is the spatial reference level. Let's identify where and how this phenomenon occur: the heart is located where the center of the acting forces equilibrates the circulatory system dynamics as a whole, maximizing better the blood flow in vessels and tissues. The heart is composed of four chambers, two atrium and two ventricles, in two pairs, left and right, each one connecting one atrium with one ventricle. Let's analyze the right pair in the understanding that the left pair is dynamically homologous. The pressure of a moving fluid, even with particles in suspension, is a function of velocity squared and twice the acceleration of earth gravity at sea level. Therefore, the heart doesn't provide to the blood flow pressure but additional velocity of an already moving flow. When the left ventricle contracts do kinetic work, then relax returning to the original departing origin, in doing so some energy is lost due to friction of internal friction in the blood, internal heart's muscle proper, which convert to heat. This heat keeps the heart warm, second to the brain as primary organs to keep life on. When the muscle of a chamber expands produce a suction effect which facilitate the incoming flow from the forces to equilibrate. In the atrium happen the same but the system is open and there is inflow instead of outflow, then its physiology is different, forces are smaller and more difficult to detect, nonetheless cannot be ignored nowadays, and our heart's assistance cannot be restricted to the ventricles. Another consideration is given to the elastic behavior of vessels by quantifying its associated elasticity coefficients, adding that coefficient to the kinetic and potential driving vein's flow, and evaluation of shock waves versus flow wave along the system to comply with due constraints at the boundaries (head, hands, and feet). Here we reach a point where considered must be given to the reaction time of the flow velocity variation when passing from steady state to unsteady state, well identified in the electrocardiogram points P and T (see FIG. 4).

Mechanics: Because each heart's chamber has a homologous Basic Unit in the device, to represent the pneumatic dynamics of each chamber, in Sheet 2 the four drawings illustrate those dynamics of a single typical chamber. Before operation: 1) Purge all air lines with all valves in open position, 2) Put the system to work in low regime for further adjustment, 3) Adjust as needed until each knob is in fix position. The open/close of each valve of the basic units are programmable managed, equally the two for systole/diastole event but only for two position: open or close. The four units have a knob for manual control each, and the left ventricle knob also is the master control for automatic adjustment of the whole system if is chosen the option of proportionality between the relationship of pressures in the four chambers as per an inequivalent healthy heart. The length of the piping from device to heart must be as short as possible, maybe not to exceed 2 ft 6 in total because the loss of head of the air stream will require extra energy and travel time can take several significant micro seconds, thou the shock wave is practically instantaneous. Once the system works as desired, the knobs are fixed with Mating Screws.

Operations: The device works, as today, to treat low pressure, for high pressure (that is to reduce pressure in the circulatory system, is recommended to prescribe medicines for the time being) also is justified as an important tool in surgery planning and its use. Data to enter: Heart Rate (beat per minute). Output reading: System Time (micro seconds) and Internal Pressures (mm Hg). Two pressures are read: the pumped air input, and real time pressure in each chamber coming from pressure sensors carrying a signal up to the Basic Unit thru the connectivity/connector. Also is read real time EKG. Beats per minute can be changed automatically by programing it as function of oxygen content in blood (%). Other rules are included in the Operators Manual. A cardiologist or qualified operator can use a device on a cart if the patient is in bed, or as a belt if waking.

Supervision and Alarms: Malfunctioning or exceeding the limits of operation as set by the cardiologist will trigger the stop of the whole device electronically. If this fail this can be made manually with the general power switch, or releasing the fastener connector by hand. It is understood that the heart can function, thou with diminished capacity, until an operator set the system in a new beginning. Not trained person can operate the device without exposing the patient to stop the heart (momentarily until disconnected) if the automatic does not work, nor the alarms sounds. The EKG is a composite of four signals, if is uniform looks like one, if is distorted some is wrong in one or more chambers, in this case can be corrected by trial and error with care watching if the guilty chamber is identified as the one failing. Otherwise can be tested using the automatic option and look if the distorted EKG go to normal, in that case the chamber unit abnormality is gone.

1) A system of six Basic Units, each one composed of two proportional valves for the systemic circuits, two proportional valves for the pulmonary circuits, two proportional valves for the systolic and diastole events, rechargeable batteries, and a micro compressor for each Basic Unit.

2) A console to manage the Basic Units of claim 1 to operate with a time precision of 1/1000 of one second.

3) An assembly to operate the system of claim as 1 a portable belt, on a cart, on a table top, or a complete portable carry on for emergencies.
